(19) United States
(12) Patent Application Publication
(10) Pub. No.: US 2008/0213407 A1
(43) Pub. Date: Sep. 4, 2008

(54) Topical application of L-arginine and menthol to increase penis size

(76) Inventors: James M. Thompson, Cincinnati, OH (US); Justin R. Thompson, Cincinnati, OH (US); Robert M. Williams, Fort Thomas, KY (US); Ronald J. Thompson, Fort Thomas, KY (US)

Correspondence Address: Donald N. Halgren, 35 Central St, Manchester, MA, 01944, US

(21) Appl. No.: 12/001,488

(22) Filed: Dec. 11, 2007

Related U.S. Application Data
(63) Continuation-in-part of application No. 11/014,429, filed on Dec. 16, 2004, now abandoned.
Application No. 11/014,429 is a continuation-in-part of application No. 11/105,228, filed on Apr. 13, 2005, now abandoned.
Application No. 11/105,228 is a continuation-in-part of application No. 11/174,137, filed on Jun. 30, 2005, now Pat. No. 7,164,469.
Application No. 11/174,137 is a continuation-in-part of application No. 11/123,595, filed on May 7, 2005.
Application No. 11/123,595 is a continuation-in-part of application No. 11/014,429, filed on Dec. 16, 2004, now abandoned.
Application No. 11/014,429 is a continuation-in-part of application No. 10/989,978, filed on Nov. 16, 2004, now abandoned.
Application No. 10/989,978 is a continuation-in-part of application No. 10/651,615, filed on Aug. 30, 2003, now Pat. No. 6,989,163.
Application No. 10/651,615 is a continuation-in-part of application No. 10/407,748, filed on Apr. 7, 2003, now abandoned.
Application No. 10/407,748 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 36/54; A61K 31/195; A61K 36/534; A61K 36/61; A61K 36/899; A61P 15/12

(52) U.S. Cl. 424/739; 514/561; 424/747; 424/742; 424/750

(57) ABSTRACT

A manual application of a topical penis sensitizing compound combination consisting of L-arginine and a cooling agent comprised of Menthol or menthol derivative or menthol analog, wherein the combination is applicable manually to the penis.

### BACKGROUND OF THE INVENTION

1. Field of the Invention

This invention relates to arrangements for increasing the size of the male penis and more particularly the topical application of specialized stimulatory medicaments. This invention relates to arrangements for the treatment or alleviation of the symptoms associated with Female Sexual Dysfunction, particularly, to the topical application of specialized stimulatory medicaments. This application is a continuation-in-part application of co-pending U.S. patent application Ser. No. 11/105,228, filed 13 Apr. 2005 (Thompson 18), and co-pending U.S. patent application Ser. Nos. 11/174,137, filed 1 Jul. 2005 (Thompson 20), 11/123,595, filed 17 May 2005 (Thompson 19), 11/014,429, filed 16 Dec. 2004 (Thompson 17), 10/989,978, filed 16 Nov. 2004 (Thompson 15), 10/651,615, filed 30 Aug. 2003 (Thompson 11A), and 10/407,748, filed 3 Sep. 2003 (Thompson 11), which are continuation-in-part of applications of co-pending U.S. patent application Ser. Nos. 10/803,148 (Thompson 4C) and 10/731,692 (Thompson 4B), both filed 9 Dec. 2003 which are divisional applications of U.S. patent application Ser. No. 10/004,091 (Thompson 4A), filed 23 Oct. 2001, now U.S. Pat. No. 6,702,733 issued 9 Mar. 2004, which is a continuation of application Ser. No. 09/520,110 (Thompson 4), filed 7 Mar. 2000, now U.S. Pat. No. 6,322,493 which is a continuation-in-part of application Ser. No. 09/469,959 (Thompson 3) filed on 21 Dec. 1999, which is a continuation-in-part of application Ser. No. 09/414,250 (Thompson 2), filed on 7 Oct. 1999, now U.S. Pat. No. 6,224,541 which is a continuation-in-part of application Ser. No. 09/340,227 (Thompson 1), filed on 1 Jul. 1999, now U.S. Pat. No. 6,179,775 each of which are incorporated herein by reference in their entirety.

2. Prior Art

In U.S. Pat. Nos. 6,322,493 and 6,702,733 there is described a combination of menthol and L-arginine, topically applied to the mucous membrane of the female clitoris, to increase vasodilatation of the corpus cavernosa. The menthol initially causes vasodilatation and secondarily functions as a lipophilic vehicle to enhance the clitoral absorption of L-arginine. The increased tissue concentrations of L-arginine cause induction of the nitric oxide synthase pathway, production of nitric oxide and cyclic-GMP, for the persistent vasodilatation of the corpus cavernosa.

The combination of the two vasodilators menthol and L-arginine, all with different methods of action for inducing vasodilatation, allows for the increase of penile arterial flow. The menthol component of our proposed combination would also act as a vehicle for the absorption of the L-arginine. L-arginine also insures a sufficient amount of substrate for supply of the nitric oxide synthase system for the production of nitric oxide and cyclic GMP, the vasodilator products of the nitric oxide synthase pathway.

### Erectile Dysfunction Synopsis

This invention is based on the discovery that effective treatment can be based on inducing the penile tissue specific expression of nitric oxide synthase, the enzyme which synthesizes the compound nitric oxide (NO), which in turn functions as a mediator of penile erection.

The physiology of normal erection can be divided into three distinct processes acting in concert: (a) increased arterial inflow; (b) decreased venous outflow; (c) active cavernosal smooth muscle relaxation. The latter appears to be the key event, but the penile blood vessel hemodynamics is also mediated by the smooth muscle of the arterial tree. Accordingly, active smooth muscle relaxation in the penile artery and sinusoids is considered to be the pivotal step in generating a normal erection. Abnormalities in penile smooth muscle function may be the critical site in erectile dysfunction

The smooth muscle relaxation of the trabeculae surrounding the lacunar spaces of the corpora cavernosa has three important functions: (a) reduction of the normally high resting (flaccid) resistance to arterial flow, thus increasing this flow through the helicine arteries into the endothelium-lined lacunar spaces; (b) regulation of blood storage into the penis, allowing penile engorgement; and (c) transmission of approximately 80% of systolic blood pressure into the cavernosal space. The latter will compress the draining venules that run in parallel between the expanding smooth muscle and the tough inelastic tunica albuginea, resulting in venous outflow restriction. Detumescence occurs by a reversal of this process, that is, an increase of the tone of the smooth muscle in both compartments leading to reduction of arterial inflow and the size of the lacunar spaces, followed by venous runoff.

The physiologic mechanism of erection of the penis involves release of nitric oxide (NO) in the corpus cavernosum during sexual stimulation. NO then activates the enzyme guanylate cyclase, which results in increased levels of cyclic guanosine monophosphate (cGMP), producing smooth muscle relaxation in the corpus cavernosum and allowing inflow of blood.

During the flaccid state the penis has a high resistance to arterial flow. A combination of menthol and L-arginine applied topically to the penis, will increase the flow of blood through dilation of the plexes in the corpus cavernosa, without specifically causing an erection. Menthol will act as a skin permeability enhancer, allowing L-arginine to enter and increase the concentration of the nitric oxide (NO) substrate within the corpus cavernosa. Increasing the concentration of NO will effectively cause smooth muscle relaxation and increased blood flow in the penis. Continuous application of this combination over an extended period of time will help to increase the efficiency of the vasculature of the corpus cavernosa, increasing size in the erect state.

### DESCRIPTION OF THE PRESENT INVENTION

The present invention relates to a hand manipulable reservoir containment fluid-dispenser arrangement for a manual discharge application of a topical preparation comprising a compound of menthol or a menthol substitute (analog or derivative) and L-arginine as that topical compound. This manually applicable compound is to be manually applied directly to a man's penis to increase vasculature efficiency and subsequently increase penis size over time.

A first preferred embodiment of a Menthol substitute may be peppermint oil. A second preferred embodiment of a Menthol substitute may be Cornmint oil. A third preferred embodiment of a Menthol substitute may be Eucalyptus oil. A fourth preferred embodiment of a Menthol substitute may be Citronella oil. A fifth preferred embodiment of a Menthol substitute may be Camphor oil. A sixth preferred embodiment of a Menthol substitute may be Cinnamon oil. A seventh preferred embodiment of Menthol would include Menthol analogs and derivatives including: (+)-neo-Menthol, Menthone, (+)-iso-Menthone, Menthyl acetate, Menthyl isovalerate, (−)-Menthyl lactate, para-Menth-1-en-3-ol, Piperitone, (−)-Menthol ethylene glycol carbonate, (−)Menthol 1- and 2-propylene glycol carbonate, (−)-Mentholel 1,2-glycerol ketal, (+)-Menthonel 1,2-glycerol ketal, and mono-Methyl succinate.

The invention thus comprises a dispensable fluid-holding reservoir for manual application of a topical penile sensitizing combination consisting essentially of L-arginine and a cooling agent comprised of Menthol, wherein the combination is applicable manually to the shaft and glands of the penis.

The cooling agent may in various preferred embodiments include peppermint oil. The cooling agent may include cornmint oil. The cooling agent may include Eucalyptus oil. The cooling agent may include Citronella oil. The cooling agent may include Camphor oil. The cooling agent may include Cinnamon oil. The cooling agent may essentially comprise peppermint oil. The cooling agent may essentially comprise cornmint oil. The cooling agent may essentially comprise Eucalyptus oil. The cooling agent may essentially comprise Citronella oil. The cooling agent may essentially comprise Camphor oil. The cooling agent essentially may comprise Cinnamon oil. The combination may contain less than 10% L-arginine. The combination may contain less than 10% menthol and the combination may contain less than 10% L-arginine. The combination may in a further embodiment be applied topically from a skin-attachable patch.

The invention thus comprises a topical sensitizing combination wherein menthol in the compound is selected from a group which comprises a Menthol Analog consisting of one or more of: (+)-neo-Menthol, Menthone, (+)-iso-Menthone, Menthyl acetate, Menthyl isovalerate, (−)-Menthyl lactate, para-menth-1-en-3ol, Piperitone, (−)-Menthol ethylene glycol carbonate, (−)-Menthol 1- and 2-propylene glycol carbonate, (−)-Menthone 1,2-glycerol ketal, (+)-Menthone 1,2-glycerol ketal, mono-Menthyl succinate, The topical sensitizing combination may contain less than 10% L-arginine. The topical sensitizing combination may contain less than 10% menthol and the combination may contain less than 10% L-arginine. The topical sensitizing combination applied to the penis increases penis size. The topical sensitizing combination applied to the penis increases penile diameter. The topical sensitizing combination applied to the penis increases penile length. The topical sensitizing combination applied to the penis increases penis surface area. The topical sensitizing combination applied to the penis increases penis volume. The topical sensitizing combination applied to the penis effectively treats premature ejaculation. The topical sensitizing combination may be topically applied from the reservoir as a cream, lotion or gel. The topical sensitizing combination may be topically applied via a transdermal patch or band, or applied via containment in a soap or shampoo.

We claim:

1. A hand manipulable reservoir containing an application of a topical penile sensitizing combination consisting essentially of L-arginine and a cooling agent comprised of Menthol, wherein said combination is applicable manually to the penis.

2. The topical sensitizing combination as recited in claim 1, wherein said cooling agent includes peppermint oil.

3. The topical sensitizing combination as recited in claim 1, wherein said cooling agent includes cornmint oil.

4. The topical sensitizing combination as recited in claim 1, wherein said cooling agent includes Eucalyptus oil.

5. The topical sensitizing combination as recited in claim 1, wherein said cooling agent includes Citronella oil.

6. The topical sensitizing combination as recited in claim 1, wherein said cooling agent includes Camphor oil.

7. The topical sensitizing combination as recited in claim 1, wherein said cooling agent includes Cinnamon oil.

8. The topical sensitizing combination as recited in claim 1, wherein said cooling agent essentially comprises peppermint oil.

9. The topical sensitizing combination as recited in claim 1, wherein said cooling agent essentially comprises cornmint oil.

10. The topical sensitizing combination as recited in claim 1, wherein said cooling agent essentially comprises Eucalyptus oil.

11. The topical sensitizing combination as recited in claim 1, wherein said cooling agent essentially comprises Citronella oil.

12. The topical sensitizing combination as recited in claim 1, wherein said cooling agent essentially comprises Camphor oil.

13. The topical sensitizing combination as recited in claim 1, wherein said cooling agent essentially comprises Cinnamon oil.

14. The topical sensitizing combination as recited in claim 1, wherein said menthol is selected from a group which comprises a Menthol Analog consisting of one or more of:
a. (+)-neo-Menthol
b. Menthone
c. (+)-iso-Menthone
d. Menthyl acetate
e. Menthyl isovalerate
f. (−)-Menthyl lactate
g. para-menth-1-en-3ol
h. Piperitone
i. (−)-Menthol ethylene glycol carbonate
j. (−)-Menthol 1- and 2-propylene glycol carbonate
k. (−)-Menthone 1,2-glycerol ketal
l. (+)-Menthone 1,2-glycerol ketal
m. mono-Menthyl succinate

15. The topical sensitizing combination as recited in claim 1, wherein said combination contains less than 10% L-arginine.

16. The topical sensitizing combination as recited in claim 1, wherein said combination contains less than 10% menthol and said combination contains less than 10% L-arginine.

17. The topical sensitizing combination as recited in claim 1, wherein said combination applied to the penis increases penis size.

18. The topical sensitizing combination as recited in claim 1, wherein said combination applied to the penis increases penile diameter.

19. The topical sensitizing combination as recited in claim 1, wherein said combination applied to the penis increases penile length.

20. The topical sensitizing combination as recited in claim 1, wherein said combination applied to the penis increases penis surface area.

21. The topical sensitizing combination as recited in claim 1, wherein said combination applied to the penis increases penis volume.

22. The topical sensitizing combination as recited in claim 1, wherein said combination applied to the penis can effectively treat premature ejaculation.

23. The topical sensitizing combination as recited in claim 1, wherein said combination may be topically applied as a cream, lotion or gel.

24. The topical sensitizing combination as recited in claim 1, wherein said combination may be topically applied via a transdermal patch or band.

25. The topical sensitizing combination as recited in claim 1, wherein said combination may be topically applied via containment in a soap or shampoo.
